(19) United States
(12) Patent Application Publication
(10) Pub. No.: US 2018/0021319 A1
(43) Pub. Date: Jan. 25, 2018

(54) COMPOSITIONS AND METHODS FOR TREATING GASTROINTESTINAL INFECTIONS

(71) Applicants: The Regents of the University of California, Oakland, CA (US); The United States Government Represented by the Department of Veterans Affairs, Washington, DC (US)

(72) Inventors: George Sachs, Encino, CA (US); Joseph R. Pisegna, Santa Monica, CA (US); David R. Scott, Calabasas, CA (US); Elizabeth A. Marcus, Sepulveda, CA (US)

(21) Appl. No.: 15/542,282

(22) PCT Filed: Jan. 8, 2016
(86) PCT No.: PCT/US2016/012592

§ 371 Date: Jul. 7, 2017

Related U.S. Application Data
(60) Provisional application No. 62/101,655, filed on Jan. 9, 2015.

(51) Int. Cl. A61K 31/4439; A61K 9/48; A61K 31/43

(52) U.S. Cl. CPC A61K 31/4439; A61K 31/43; A61K 9/48

(57) ABSTRACT

The invention described herein provides compositions and methods for treating or preventing Helicobacter pylori infection.

### RELATED APPLICATION

This application claims the benefit of priority to U.S. Provisional Patent Application serial number 62/101,655, filed Jan. 9, 2015. This application is hereby incorporated herein by reference in its entirety.

### GOVERNMENT SUPPORT

This invention was made with Government support under DK053642, awarded by the National institutes of Health. The Government has certain rights in the invention. This work was supported by the U.S. Department of Veterans Affairs, and the Federal Government has certain rights in the invention.

### BACKGROUND OF THE INVENTION

The pathogen, Helicobacter pylori, a small, spiral, Gram-negative, microaerophilic bacterium is found in the stomach of about 50% of the world's population. H. pylori is the only known organism to colonize the normal acid-secreting human stomach by infiltrating the mucous layer overlying the gastric epithelial cells. Colonization is associated with several gastric diseases, including gastritis, peptic and duodenal ulcers, gastric carcinoma and MALT lymphoma. The International Agency for Research on Cancer (IARC) has classified the organism as a type 1 carcinogen. Although certain findings suggest that H. pylori infection may have a beneficial effect on gastroesophageal reflux disease (GERD) and other nongastric manifestations of infection, the carcinogenic nature of this bacterium partially drives the need for a therapeutic.

Standard therapy to eradicate H. pylori necessitates a complicated regimen of at least two antibiotics and stomach acid suppression. Current methods for eradication of this organism call for using proton pump inhibitors, including the commercial class omeprazole, esomeprazole, pantoprazole, lansoprazole, rabeprazole and dexlansoprazole, in combination with amoxicillin and clarithromycin. However, antibiotic resistance to clarithromycin has made successful treatment and eradication progressively more difficult. Indeed, the standard therapy currently provides unacceptably low treatment success and a low likelihood of eradication of H. pylori.

There is a long-felt need in the art for providing compositions and related methods for treating and/or preventing H. pylori infection without promoting antibiotic resistance.

### SUMMARY OF INVENTION

In one aspect, the invention relates to a composition comprising an antibiotic and a compound having the structure:

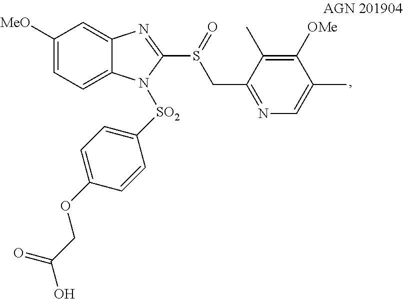 [SMILES: COc1ccc2c(c1)nc(S(=O)Cc1ncc(C)c(OC)c1C)n2S(=O)(=O)c1ccc(OCC(=O)O)cc1]

or a salt, ester, or prodrug thereof. In certain embodiments, the antibiotic is a β-lactam antibiotic. In certain embodiments, the antibiotic is amoxicillin. In certain embodiments, the composition is provided as a capsule formulated for simultaneous release of the compound and the antibiotic.

In another aspect, the invention relates to a method of treating or preventing a Helicobacter pylori infection in a subject in need thereof, comprising: conjointly administering to the subject an antibiotic and a compound having the structure:

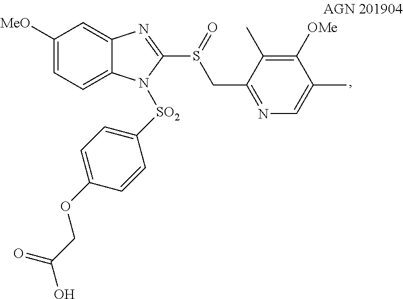 [SMILES: COc1ccc2c(c1)nc(S(=O)Cc1ncc(C)c(OC)c1C)n2S(=O)(=O)c1ccc(OCC(=O)O)cc1]

or a salt, ester, or prodrug thereof. In certain embodiments, the antibiotic is a β-lactam antibiotic. In certain embodiments, the antibiotic is amoxicillin. In certain embodiments, the compound and the antibiotic are administered simultaneously. In certain embodiments, the antibiotic and the compound are administered in a capsule formulated for simultaneous release of the compound and the antibiotic.

In yet another aspect, the invention provides a composition for use to treat or prevent a Helicobacter pylori infection in a subject in need thereof, comprising an antibiotic and a compound having the structure:

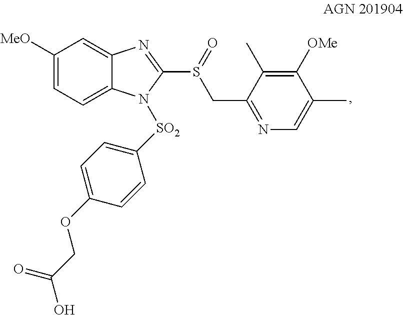 [SMILES: COc1ccc2c(c1)nc(S(=O)Cc1ncc(C)c(OC)c1C)n2S(=O)(=O)c1ccc(OCC(=O)O)cc1]

or a salt, ester, or prodrug thereof.

### DETAILED DESCRIPTION

The present application discloses a composition and a method to treat Helicobacter pylori infection. The present application derives from the unexpected finding that AGN 201904 can be used with amoxicillin alone (i.e., dual therapy) to successfully treat a H. pylori infection. This dual therapy had the unexpected result of a higher eradication rate and no H. pylori resistance as compared to standard H. pylori infection therapies.

In one aspect, the invention relates to a method of treating or preventing a Helicobacter pylori infection in a subject in need thereof, comprising: conjointly administering to the subject an antibiotic and a compound having the structure:

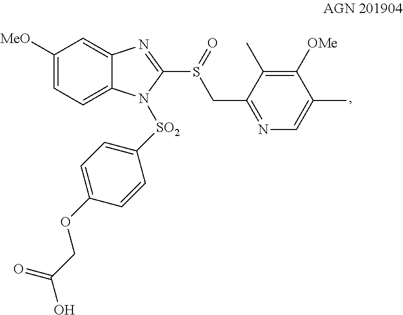 [SMILES: COc1ccc2c(c1)nc(S(=O)Cc1ncc(C)c(OC)c1C)n2S(=O)(=O)c1ccc(OCC(=O)O)cc1]

or a salt, ester, or prodrug thereof.

In certain embodiments, the invention relates to a composition for use to treat or prevent a Helicobacter pylori infection in a subject in need thereof, comprising an antibiotic and a compound having the structure:

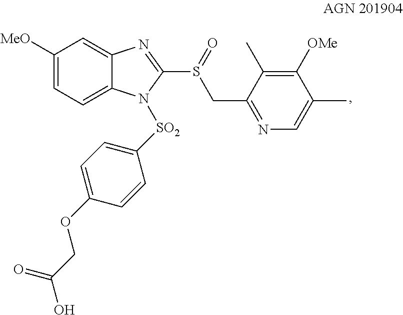 [SMILES: COc1ccc2c(c1)nc(S(=O)Cc1ncc(C)c(OC)c1C)n2S(=O)(=O)c1ccc(OCC(=O)O)cc1]

or a salt, ester, or prodrug thereof.

In certain embodiments, the antibiotic is a β-lactam antibiotic. If desired, a suitable derivative of a beta-lactam antibiotic may also be used. Non-limiting examples of suitable derivatives include prodrugs, metabolites, esters, ethers, hydrates, polymorphs, solvates, complexes, enantiomers, adducts and the like of such beta-lactam antibiotics. Non-limiting examples of typical beta-lactam antibiotics include those belonging to penicillins, penems, carbapenems, cephalosporins, and monobactams. Typical examples of beta-lactam antibiotics include, but are not limited to amoxicillin, ampicillin, azidocillin, azlocillin, aztreonam, bacampicillin, benzathine, benzathine phenoxymethylpenicillin, benzylpenicillin, biapenem, carbacephem, carbenicillin, carumonam, cefacetrile, cefaclor, cefadroxil, cefalexin, cefaloglycin, cefalonium, cefaloridine, cefalotin, cefamandole, cefapirin, cefatrizine, cefazaflur, cefazedone, cefazolin, cetbuperazone, cefcapene, cefdaloxime, cefdinir, cefditoren, cefepime, cefetamet, cefixime, cefmenoxime, cefmetazole, cefminox, cefodizime, cefonicid, cefoperazone, ceforanide, cefotaxime, cefotetan, cefotiam, cefovecin, cefoxitin, cefozopran, cefpimizole, cefpiramide, cefpirome, cefpodoxime, cefprozil, cefquinome, cefradine, cefroxadine, cefsulodin, ceftaroline fosamil, ceftazidime, cefteram, ceftezole, ceftibuten, ceftiofur, ceftiolene, ceftizoxime, ceftobiprole, ceftolozane, ceftriaxone, cefuroxime, cefuzonam, cephamycin, clometocillin, cloxacillin, dicloxacillin, doripenem, epicillin, ertapenem, faropenem, flomoxef, flucloxacillin, hetacillin, imipenem, latamoxef, loracarbef, mecillinam, meropenem, metampicillin, methicillin, mezlocillin nafcillin oxacephem, oxacillin, panipenem, penamecillin, pheneticillin, piperacillin, pivampicillin, procaine benzylpenicillin, propicillin, sulbenicillin, tabtoxin, talampicillin, temocillin, ticarcillin, tigemonam, and the like.

In certain embodiments, the invention relates to a composition comprising an antibiotic and AGN 201904. In certain embodiments, the invention relates to a composition comprising an antiobiotic and AGN 201904 and a pharmaceutically acceptable carrier.

Patients, including but not limited to humans, can be treated by administering to the patient an effective amount of the active compound(s) or a pharmaceutically acceptable prodrug or salt thereof in the presence of a pharmaceutically acceptable carrier or diluent. The active materials can be administered by any appropriate route, for example, orally, parenterally, intravenously, intradermally, subcutaneously, or topically, in liquid or solid form.

The concentration of active compound(s) in the drug composition will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient can be administered at once, or can be divided into a number of smaller doses to be administered at varying intervals of time.

In certain embodiments, the mode of administration of the active compound(s) is oral. Oral compositions will generally include an inert diluent or an edible carrier. They can be enclosed in gelatin capsules or compressed into tablets. For the purpose of oral therapeutic administration, the active compound(s) can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, unit dosage forms can contain various other materials that modify the physical form of the dosage unit, for example, coatings of sugar, shellac, or other enteric agents.

The compound can be administered as a component of an elixir, suspension, syrup, wafer, chewing gum or the like. A syrup can contain, in addition to the active compound(s), sucrose or sweetener as a sweetening agent and certain preservatives, dyes and colorings and flavors.

The compound or a pharmaceutically acceptable prodrug or salts thereof can also be mixed with other active materials that do not impair the desired action, or with materials that supplement the desired action, such as antibiotics, antifungals, anti-inflammatories or other antivirals, including but not limited to nucleoside compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents, such as ethylenediaminetetraacetic acid; buffers, such as acetates, citrates or phosphates, and agents for the adjustment of tonicity, such as sodium chloride or dextrose. The parental preparation can be enclosed in ampoules, disposable syringes or multiple dose vials made of glass or plastic.

If administered intravenously, carriers include physiological saline and phosphate buffered saline (PBS).

In certain embodiments, the active compound(s) are prepared with carriers that will protect the compound against rapid elimination from the body, such as a controlled release formulation, including but not limited to implants and microencapsulated delivery systems. Biodegradable, biocompatible polymers can be used, such as ethylene vinyl acetate, polyanhydrides, polyglycolic acid, collagen, polyorthoesters and polylactic acid. For example, enterically coated compounds can be used to protect cleavage by stomach acid. Methods for preparation of such formulations will be apparent to those skilled in the art. Suitable materials can also be obtained commercially.

Liposomal suspensions (including but not limited to liposomes targeted to infected cells with monoclonal antibodies to viral antigens) are also preferred as pharmaceutically acceptable carriers. These can be prepared according to methods known to those skilled in the art, for example, as described in U.S. Pat. No. 4,522,811 (incorporated by reference). For example, liposome formulations can be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound(s) is then introduced into the container. The container is then swirled by hand to free lipid material from the sides of the container and to disperse lipid aggregates, thereby forming the liposomal suspension.

The present invention also provides a composition comprising the compound AGN201904 and antibiotic compositions described herein. The composition may be a suitable pharmaceutical composition comprising suitable carriers or excipients.

The compositions and methods of the present invention may be utilized to treat a subject in need thereof. In certain embodiments, the subject is a mammal such as a human, or a non-human mammal. When administered to an animal, such as a human, the composition is preferably administered as a pharmaceutical composition comprising, for example, a composition of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters. In preferred embodiments, when such pharmaceutical compositions are for human administration, e.g., for parenteral administration, the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, powder, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize or to increase the absorption of a composition of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The pharmaceutical composition (preparation) also can be a liposome or other polymer matrix, which can have incorporated therein, for example, a composition of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose: (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compositions may also be formulated for inhalation. In certain embodiments, a composition may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

In certain embodiments, a single capsule may be used at each administration because a single capsule reproducibly provides simultaneous release of the antibiotic and AGN 201904. In certain embodiments, the capsule is formulated such that the antibiotic (e.g., amoxicillin) is absorbed rapidly along with the absorption of AGN 201904 and with greater gastric acid inhibition. In certain embodiments, near simultaneous release is achieved by administering all components of the invention as a single pill or capsule.

In certain embodiments, compounds of the invention may be used alone or conjointly administered with another type of therapeutic agent (e.g., an antibiotic). As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of compounds of the invention with one or more additional therapeutic agent(s) (e.g., one or more additional antibiotic agent(s)) provides improved efficacy relative to each individual administration of the compound of the invention or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of
active ingredient that can be combined with a carrier material to produce a single dosage form will generally be that amount of the antibiotic or compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

In some embodiments of the present invention, the composition that is suitable for use in the invention may be administered orally, topically or parenterally.

Dosage forms for the topical or transdermal administration include powders, sprays, ointments, pastes, creams, lotions, gels, solutions, patches and inhalants. The composition may be mixed under sterile conditions with a pharmaceutically acceptable carrier, and with any preservatives, buffers, or propellants that may be required.

The ointments, pastes, creams and gels may contain, in addition to an antibiotic, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the antibiotic or compound, excipients such as lactose, talc, silicic acid, aluminum hydroxide, calcium silicates and polyimide powder, or mixtures of these substances. Sprays can additionally contain customary propellants, such as chlorofluorohydrocarbons and volatile unsubstituted hydrocarbons, such as butane and propane.

The composition of the invention may be formulated with an excipient and component that is common for such oral compositions or food supplements, e.g., especially fatty and/or aqueous components, humectants, thickeners, preserving agents, texturizers, flavor enhancers and/or coating agents, antioxidants and preserving agents. Formulating agents and excipients for oral compositions, and especially for food supplements, are known in this field and will not be the subject of a detailed description herein.

In the case of a composition in accordance with the invention for oral administration, the use of an ingestible support is preferred. The ingestible support may be of diverse nature according to the type of composition under consideration. Tablets, gel capsules or lozenges, suspensions, oral supplements in dry form and oral supplements in liquid form are especially suitable for use as food supports.

Formulation of the oral compositions according to the invention may be performed via any usual process known to those skilled in the art for producing drinkable solutions, sugar-coated tablets, gel capsules, gels, emulsions, tablets to be swallowed or chewed, wafer capsules, especially soft or hard wafer capsules, granules to be dissolved, syrups, solid or liquid foods, and hydrogels allowing controlled release. Formulation of the oral compositions according to the invention may be incorporated into any form of food supplement or enriched food, for example food bars, or compacted or loose powders. The powders may be diluted with water, with soda, with dairy products or soybean derivatives, or may be incorporated into food bars.

In some embodiments, the composition according to the invention administered orally may be formulated in the form of sugar-coated tablets, gel capsules, gels, emulsions, tablets, wafer capsules, hydrogels, food bars, compacted or loose powders, liquid suspensions or solutions, confectioneries, fermented milks, fermented cheeses, chewing gum, toothpaste or spray solutions.

An effective amount of the composition may be administered in a single dose per day or in fractional doses over the day, for example two to three times a day. By way of example, the administration of a composition according to the invention may be performed at a rate, for example, of 3 times a day or more, generally over a prolonged period of at least a week, 2 weeks, 3 weeks, 4 weeks, or even 4 to 15 weeks, optionally comprising one or more periods of stoppage or being repeated after a period of stoppage.

As one of skill in the art will appreciate, compositions of the present invention, not having adverse effects upon administration to a subject, may be administered daily to the subject,

Preferred embodiments of this invention are described herein. Of course, variations, changes, modifications and substitution of equivalents of those preferred embodiments will become apparent to those of ordinary skill in the art upon reading the foregoing description. The inventors expect skilled artisans to employ such variations, changes, modifications and substitution of equivalents as appropriate, and the inventors intend for the invention to be practiced otherwise than specifically described herein. Those of skill in the art will readily recognize a variety of non-critical parameters that could be changed, altered or modified to yield essentially similar results. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

While each of the elements of the present invention is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

### Definitions

For purposes of the present invention, the following definitions will be used (unless expressly stated otherwise).

As used herein, the term “administering” means the actual physical introduction of a composition into or onto (as appropriate) a subject. Any and all methods of introducing the composition into subject are contemplated according to the invention; the method is not dependent on any particular means of introduction and is not to be so construed. Means of introduction are well-known to those skilled in the art, and also are exemplified herein.

As used herein, the term “beta-lactam antibiotic” refers to a compound with antibiotic properties and containing a beta-lactam ring in their molecular structure.

As used herein, the terms “effective amount”, “effective dose”, “sufficient amount”, “amount effective to”, “therapeutically effective amount” or grammatical equivalents thereof mean a dosage sufficient to produce a desired result, to ameliorate, or in some manner, reduce a symptom or stop or reverse progression of a condition and provide either a subjective relief of a symptom(s) or an objectively identifiable improvement as noted by a clinician or other qualified observer. Amelioration of a symptom of a particular condition by administration of a pharmaceutical composition described herein refers to any lessening, whether permanent or
temporary, lasting, or transitory, that can be associated with the administration of the pharmaceutical composition.

As used herein, the term “prodrug” is intended to encompass compounds which, under physiologic conditions, are converted into the therapeutically active agents of the present invention. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds in a formulation represented above can be replaced with the corresponding suitable prodrug, e.g., wherein a hydroxyl in the parent compound is presented as an ester or a carbonate or carboxylic acid present in the parent compound is presented as an ester.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia
for use in animals, and more particularly in humans.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, a “subject” means a human or animal (in the case of an animal, more typically a mammal). In one aspect, the subject is a human.

As used herein, the term “treating” is art-recognized and includes administration to the host of one or more of the subject compositions, e.g., to diminish, ameliorate, or stabilize the existing unwanted condition or side effects thereof.

### Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

### Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A composition comprising an antibiotic and a compound having the structure: 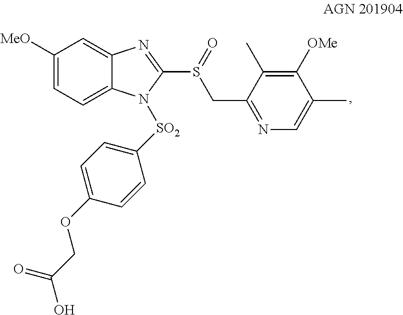 [SMILES: COc1ccc2c(c1)nc(S(=O)Cc1ncc(C)c(OC)c1C)n2S(=O)(=O)c1ccc(OCC(=O)O)cc1]
or a salt, ester, or prodrug thereof.

2. The composition according to claim 1, wherein the antibiotic is a β-lactam antibiotic.

3. The composition according to claim 2, wherein the β-lactam antibiotic is amoxicillin.

4. The composition according to claim 1, wherein the composition is provided as a capsule formulated for simultaneous release of the compound and the antibiotic.

5. A method of treating or preventing a Helicobacter pylori infection in a subject in need thereof, comprising: conjointly administering to the subject an antibiotic and a compound having the structure: 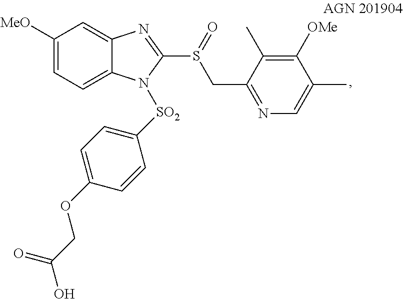 [SMILES: COc1ccc2c(c1)nc(S(=O)Cc1ncc(C)c(OC)c1C)n2S(=O)(=O)c1ccc(OCC(=O)O)cc1]
or a salt, ester, or prodrug thereof.

6. The method according to claim 5, wherein the antibiotic is a β-lactam antibiotic.

7. The method according to claim 6, wherein the β-lactam antibiotic is amoxicillin.

8. The method according to claim 5, wherein the compound and the antibiotic are administered simultaneously.

9. The method of claim 8, wherein the antibiotic and the compound are administered in a capsule formulated for simultaneous release of the compound and the antibiotic.

10. (canceled)
